(19) United States
(12) Patent Application Publication
(10) Pub. No.: US 2025/0114516 A1
(43) Pub. Date: Apr. 10, 2025

(54) Intravenous Line Electrical Connector Assemblies, Networks and Associated Methods

(71) Applicant: Convergence Medical Sciences Ltd, Calgary (CA)

(72) Inventor: Steven Daniel ROY, Calgary (CA)

(21) Appl. No.: 18/727,862

(22) PCT Filed: Jan. 11, 2023
(86) PCT No.: PCT/CA2023/050027

§ 371 Date: Jul. 10, 2024

Related U.S. Application Data
(60) Provisional application No. 63/298,710, filed on Jan. 12, 2022.
(60) Provisional application No. 63/313,411, filed on Feb. 24, 2022.

(51) Int. Cl. A61M 5/14; A61M 5/142; A61M 5/172; A61M 39/10

(52) U.S. Cl. CPC A61M 5/1413; A61M 5/1409; A61M 5/142; A61M 5/172; A61M 39/10; A61M 2005/1403; A61M 2039/1022; A61M 2039/1038; A61M 2205/60

(57) ABSTRACT

An intravenous line connector assembly has a first and second connectors, each having a lumen and an electrical terminal. When the first connector is connected to the second connector, the first lumen is connected to the second lumen to allow fluid communication between the first and second connectors, and the first terminals are connected with the second terminals to allow electrical signals to pass between the first and second connectors. These connectors allow an intravenous line network to be formed which carried both fluids and electrical signals. The electrical signals can then be used to communicate between components of the intravenous line network to help improve the care of the patient.

### CORRESPONDING APPLICATIONS

This claims priority to U.S. Provisional Application 63/298,710 filed on Jan. 12, 2022 and U.S. Provisional Application 63/313,411 filed on Feb. 24, 2022. The contents of both priority applications are hereby incorporated by reference in their entirety.

### TECHNICAL FIELD

The present disclosure relates to an intravenous (IV) line connector assembly configured to allow fluids and electrical signal to be transmitted. In particular, the present disclosure relates to using these connector assemblies to form an electrical network to allow power and/or data to be transmitted to electrical equipment associated with the IV lines.

### BACKGROUND

Intravenous fluids, such as saline solution, blood, or other fluid, is typically supplied from a syringe or a bag that is hung from a hanger close to the patient. The fluid is delivered through a series of IV lines connected in series using connector assemblies. In addition, a common IV line may be branched to allow fluids from multiple sources to be injected into the patient without the need for multiple intravenous cannulas

In ongoing care situations, an IV system may be associated with multiple pieces of electrical equipment such as pumps. The configuration of the various components of the fluid network affects how fluid are administered to the patient. For example, multiple fluids injected via a common line must be managed so that incompatible fluids are not present in the common line simultaneously, and that the total fluid flow through the common line does not exceed the capacity of the line.

Currently IV pumps can be used to identify possible faults in the IV lines and to generate alarms to notify medical staff.

One of the most common infusion pump alarms is for an occlusion in the IV line. This can be caused air in the IV, a clot or bend in the cannula, by the patient kinking the line by bending an arm, or catching the loose tubing on the bed.

Many of these alarms are false and dealing with the alarms can be a major issue, particularly where the patient is in medical isolation and there is a lengthy procedure for medical staff to enter the room to deal with the alarms.

Medical isolation also makes it difficult to perform routine adjustments to the IV equipment, such as adjusting the pump speed. For example, for some patients, the medical staff have to constantly adjust medications to maintain a delicate balance (for example, trying to balance keeping the blood pressure up to perfuse the organs but not too high as to cause bleeding).

### SUMMARY

In accordance with the present disclosure, there is provided an intravenous line connector assembly comprising:
a first connector comprising a first lumen and one or more electrical first terminals; and
a second connector comprising a second lumen and one or more electrical second terminals,
wherein when the first connector is connected to the second connector, the first lumen is connected to the second lumen to allow fluid communication between the first and second connectors, and the first terminals are connected with the second terminals to allow electrical signals to pass between the first and second connectors.

By having first and second connectors corresponding to first and second lumen, a wired electrical network may be established with network topology which matches that of the fluid network.

A terminal may be an electrically conducting component of the connector having an exposed surface for connection to another terminal to form an electrical connection. The electrical terminal may be connected to an electrical cable or wire to facilitate the transfer of the electrical signal elsewhere via a wired connection. A connector may comprise multiple terminals, each terminal configured to carry, or be connected to, a different electrical signal (e.g., earth, ground, direct current, alternating current, live, positive, negative and/or data).

A connector may comprise multiple terminals configured to carry the same signal. For example, a first connector may comprise one first terminal configured to be connected to ground, and two separate first terminals configured to be connected to the same data signal. It will be appreciated that, in this case, the second connector may have one or more corresponding terminals to connect to one or both of the data signal first terminals.

The first and second connectors may comprise respective complementary engagement surfaces. One or more of the engagement surfaces may be configured to physically maintain connection between the first and second connectors. One or more of the engagement surfaces may be configured to restrict how the first and second connectors engage with each other. For example, the engagement surfaces may be configured such that each first terminal can only engage with a corresponding second terminal. The engagement surfaces may be configured such that when the engagement surfaces of the first and second connectors are engaged, the first terminals and corresponding second terminals are always engaged.

The complementary engagement surfaces may comprise screw threads. In this case, the engagement surfaces may act to physically maintain connection (i.e., by tightening the screw).

The screw threads may comprise different thread starts corresponding to different first and second terminals. In this case, the engagement surfaces may act to restrict how the first and second connectors engage with each other. For example, when the threads start to engage, the first terminal thread projection can only be connected to a corresponding second terminal thread recess as the screw is tightened-it can not switch to a second terminal thread recess as the first and second terminals are rotated with respect to each other.

At least one of the first terminals may be configured to connect to a corresponding said second terminal via the screw threads.

One of the first and second connectors may comprise a rotatable sleeve which is rotatable independently of the lumen, the rotatable sleeve comprising engagement surfaces for physically connecting with the other connector.

At least one of the first terminals may be formed on a surface which is rotationally invariant about the lumen axis.

The intravenous line connector assembly may comprise two electrical first terminals and two electrical second terminals.

The intravenous line connector assembly may comprise three electrical first terminals and three electrical second terminals.

At least one of the first and second connectors may have no moving parts.

For one or both of the connectors in a connector assembly, the electrical terminal may be rigidly fixed with respect to the lumen.

The lumen of a connector may be formed from an electrically insulating material.

The intravenous line connector assembly may comprise an intravenous line connected to at least one of the first and second connectors.

An intravenous (IV) line may comprise intravenous tubing. Intravenous tubing is typically used to connect the bag of medication to the patient, and is either gravity driven or forced by a pump calibrated to give the fluids over a longer period. Intravenous tubing is typically a transparent flexible tube formed from, for example, PVC, polyethylene, or polypropylene plastic. IV tubing may be between 20 cm and 2 m in length. IV tubing may have an inner bore of less than 10 mm. IV tubing may have an inner bore of greater than 0.5 mm.

The electrical signals may comprise power signals. The electrical signals may comprise data signals. The data signals may include information on one or more of the following: the type of device (e.g., catheter, pump); a unique identifier of the device (e.g., which may be used to determined the type and/or age of device); fluid information (e.g. the fluid contained in the syringe); and flow capacity (e.g. catheter bore).

According to a further aspect, there is provided an intravenous line network comprising:
multiple intravenous components and/or lines, each intravenous line comprising at least one electrical wire;
an intravenous line connector assembly according to claim 1 connecting the multiple intravenous lines; and
electrical equipment electrically connected to at least one of the electrical wires.

The intravenous line network may comprise at least three intravenous components.

The electrical equipment may be configured to interact with the fluids passing through the multiple intravenous lines. For example, the electrical equipment may comprise one or more of: a pump configured to pump the fluid, a flow meter configured to measure the flow rate, and a volume meter configured to measure the volume of fluid injected.

The intravenous line network may comprise multiple fluid sources connected to a common intravenous line, and wherein the electrical equipment comprises:
a respective data source associated with each fluid source, the data source being configured to transmit fluid identity data identifying the respective fluid; and
a controller configured to receive the fluid identity data transmitted from each data source via the electrical wires in the intravenous lines and the electrical connections provided by the intravenous line connector assemblies.

The controller may be configured to identify whether or not the fluids are compatible. In particular, Y-site compatibility relates to whether two fluids are compatible when being introduced a common line (usually via a Y-site or Y-branch).

The controller may be configured, in response to identifying an incompatibility between two or more of the fluids, to create a flushing protocol to avoid the incompatible fluids coming in contact within the intravenous line network.

The electrical equipment may comprise a controller, the controller being configured to:
record a time at which one or more of the components of the intravenous line network is first connected, and
calculate the age of the components based on the time elapsed between first connection and the current time.

The electrical equipment may comprise a controller, and multiple pumps delivering fluid from multiple fluid sources, the controller being configured to switch between pumps and fluid sources based on when the fluid source is exhausted.

The electrical equipment may comprise a controller, and a data source configured to communicate the size of a lumen to the controller, the lumen receiving fluids from multiple fluid sources, the controller configured to adjust the flow rates being delivered by multiple fluid sources such that the total flow rate delivered to the lumen is below a predetermined threshold.

The electrical equipment may comprise a controller, the controller being configured to allow alarms to be acknowledged remotely.

Screw threads may be configured to allow only one orientation of the screw. For example, the screw threads may comprise multiple starts, where the cross section of at least one of the starts is unique (e.g., in terms of size and/or shape). This means that the screw threads can only be connected in one way.

Each intravenous lines may have an electrical wire embedded within the fluid channel wall or attached to the outside of the fluid channel within an electrically insulating sheath. This means that as the fluid line is bent and shaped, the electrical wires bend with the fluid line.

An intravenous line may have electrical wire along at least 50% of the length of the fluid line. An intravenous line may have electrical wire along the entire length of the fluid line. The electrical wire may comprise an electrically insulated sheath.

The intravenous line may have terminals or electrical connectors along the length of the intravenous line, the terminals allowing electrical components to be connected to the electrical network. The terminals may be in the form of a socket configured to receive a plug to connect to the electrical components.

The intravenous line connector assembly may be configured to connect two IV lines together. A component may comprise multiple intravenous line connector assemblies with interconnected lumens to provide a Y-branch or manifold connector configured to join three or more IV lines together.

The intravenous line connector assembly may comprise releasably connectable connectors. Releasably connectable connectors may be connectors which can be repeatedly connected and separated without damaging the connectors. Releasably connectable connectors may be connectable and separable by hand (i.e., without the use of tools).

An intravenous component may comprise a lumen for passing fluid within an IV fluid network. The intravenous component may be, or be directly connectable to, an intravenous line. An intravenous component may comprise one or more of: a fluid source (e.g., an IV bag), a cannula, an intravenous line, a pump (e.g., a syringe pump, an infusion pump), a drip chamber, a fluid connector (e.g., a straight connector, a Y connector).

An intravenous component may comprise a data source (e.g., in the form of a data tag). The data tag may comprise memory connected to the wires within the intravenous component so that data may be transmitted via an intravenous wired network formed by the connected connector assemblies. The data source may be embedded within the intravenous component. The data source may comprise identity data. The data source may be readable by the controller. The data source may transmit data over the wired network to the controller. The data source may comprise memory and be connected to the wired network.

Within an intravenous network, a plurality of the intravenous components may use a common connector shape (e.g., Luer locks) such that the components may be connected together in a variety of different ways. In some embodiments, particular connectors may have a particular shape to ensure that they can only be connected to compatible equipment. For example, a drip chamber may have a different shape so that it can only be connected to a liquid source.

The intravenous line network may form part of a patient care system. The patient care system may comprise external electronics such as data recorders and patient sensors (e.g., blood pressure monitors, heartbeat monitors etc.).

The controller may be configured to: receive data via the electrical wires relating to the type of the multiple intravenous components (e.g., each of the multiple intravenous components); and, based on the received intravenous component types, determine a network topology for the fluid network.

The controller may be configured to: receive data via the electrical wires relating to the type of the multiple intravenous components (e.g., each of the multiple intravenous components). Knowledge of the type or identity of the components connected to an IV network can be helpful to medical staff in, for example, determining which components belong to which network (i.e., even without knowing the precise topology of the network). Likewise, knowledge of the components of the network can be useful in controlling the network, even if the topology is not known. For example, knowledge of which fluid sources are attached to which cannula allow determinations of compatibility to be made, regardless of the precise configuration of IV lines between the sources and cannula.

The intravenous line network may be configured to provide power over IV (e.g., to power small patient monitors).

The intravenous line network may be configured to facilitate battery equalizing or prioritizing (e.g., recharging a pump running a lifesaving drug from a pump running a lower priority medication).

The intravenous line network may be configured to facilitate automatic identification of all connected medications (whether IV pumps or single use syringes).

An IV line may comprise cables (i.e., electrical wires) configured to attach to a connector to form an electrical connection with each of the terminals of the attached connector. The cables may be embedded in the wall of the line, or along the outside of the line along the cable's length. The cables may extend along the length of the line (e.g., or along at least 70% of the IV line's length).

The patient care system may be configured to detect drug incompatibilities (e.g., +/−mitigation).

The controller may be configured to provide software updates to electronic equipment (e.g., IV pumps) over the wired network. The system may provide more efficient wide area network (WAN) communication (e.g., one pump downloads, updates and installs, checks result, pushes update if successful, reverts using neighbour pump if fails).

The system may control all the connected pumps (e.g., acknowledge alarms, change infusion rates, pause infusions, etc.). This is extremely useful for a patient in isolation (using an extension to outside of the room) or in settings with restricted movement (emergency transport in small airframes).

The system may detect disconnection from patient (and/or pausing pump).

A pump may form part of an electronic infusion device (EID). The pump and/or EID may a variety of safety features including, for example, one or more of: alerts for air and occlusions, a medication administration library, the ability to calculate infused volumes, and back up battery power.

The system may detect IV medication administration (and potentially auto-flushing after IV push).

The system may detect IV cannula size in the patient (and maximum total infusion rate of connected pumps) may help prevent interstitial IVs.

The system may detect IV cannula age (e.g., by imprinting with current date/time on first connection).

The system may be configured to switch between fluids (when one IV bag is empty, stop the pump and continue with the next bag of the same medication on a second pump).

The system may be configured to run incompatible medications together through intelligent programming (e.g., instead of running both meds at 40 mL/h+100 mL/h runner, the system may run 2 mL of the first medication over 30 seconds, flush 2.5 mL runner over 60 seconds, run 2 mL of the second medication over 30 seconds, flush with 2.5 mL runner over 30 seconds, repeat cycle continuously).

The system may not require wireless communication (which may improve security as a hacker can not intercept or inject wireless signals). That is, the communication may be entirely via wired connections.

The lumen may be formed by the walls of a channel. A lumen in the context of this disclosure is the bore of a channel or tube (e.g., as of a hollow needle or catheter).

A computer program may be stored on a non-transitory medium such as a CD or a DVD. A computer program may be stored on a USB or other local solid-state disk or in the cloud. Computer programs may be configured to carry out one or more of the methods disclosed herein.

The controller may comprise a processor and memory. The memory may store computer program code. The processor may comprise, for example, a graphics processing unit, a central processing unit, a microprocessor, an application-specific integrated circuit (or ASIC) or a multicore processor. The memory may comprise, for example, flash memory, a hard-drive, volatile memory.

The controller may be a central controller configured to coordinate and control other elements in the IV electrical network.

The controller may comprise distributed controller. A distributed controller may comprise multiple pieces of electrical components networked together, e.g., in a “mesh” or “swarm” type network. Such a network may be non-hierarchical. For example, the pumps may be configured to operate alone, paired, or in a large network. The user could interact with the network via one of the electrical components which has a user interface (e.g., one of the pumps).

For example, the electrical equipment comprises multiple pumps. At least one of the pumps may be a controller pump having a controller configured to control at least one of the other said pumps. This would allow the multiple pumps to be controlled from one pump. The instructions may be generated by a user and/or by the pump itself. For example, the controller pump may be configured to achieve a predetermined effect, such as ensuring that the aggregate pump rate is below a certain threshold, or to run a predetermined flushing protocol between injecting incompatible medications through the same cannula. Controlling another pump may include turning it on and off and/or adjusting the pumping rate.

### BRIEF DESCRIPTION OF THE DRAWINGS

Various objects, features and advantages of the present disclosure will be apparent from the following description of particular embodiments of the present disclosure, as illustrated in the accompanying drawings. The drawings are not necessarily to scale, emphasis instead being placed upon illustrating the principles of various embodiments of the present disclosure. Similar reference numerals indicate similar components.

FIG. 1 is a cut through view of a first embodiment of an intravenous line connector assembly in an unconnected configuration.

FIG. 2a is a cut through view of a further embodiment of an intravenous line connector assembly in an unconnected configuration.

FIG. 2b is a schematic view of a rotatable connector assembly used as part of the embodiment of FIG. 2a.

FIG. 3 is a front view of a first embodiment of an intravenous line network.

FIG. 4 is a cut through view of a further embodiment of an intravenous line connector assembly in an unconnected configuration.

FIG. 5a is a front view of a further embodiment of an intravenous line network.

FIG. 5b is a determined topology for the intravenous line network of FIG. 5b.

### DETAILED DESCRIPTION

### Introduction

As described in the Background section, IV lines are commonly used in medical situations to provide various fluids to a patient. It is not uncommon for a patient to receive multiple fluids from multiple sources over a period of time. This may involve the use of pumps to control and automate the delivery of fluids. In addition, to reduce the number of injection sites, it is also common for multiple fluids to be delivered to the patient via a common needle or cannula.

The inventors have realised that although there may be a connected fluid network of lines (e.g., connected to the common needle), the electrical equipment associated with the patient typically operates autonomously and independently of other electrical equipment. For example, each pump controlling the fluid flow from a fluid bag (e.g., of saline solution or medication), is set manually using the user interface of that particular pump. This works well where the situation is simple (e.g., a small number of fluids being administered) and stable (e.g., where the same fluids are administered over an extended period of time). It will be appreciated that where the situation is more complex (e.g., there are a large number of different fluids being administered), and rapidly developing, it may be more difficult for the doctor or nurse to set up or adapt the fluid network to optimize the treatment to the patient.

The inventors have realised that one solution would be to use the IV lines to form a corresponding electrical network to allow the various electrical components associated with the fluid network to communicate.

The use of a wired network may have several advantages over a wireless network including:
A wired network is more difficult to hack and so provides greater privacy for the patient.

1A wired network in tandem with the fluid network means that the electrical equipment in the fluid network can communicate with other electrical equipment in the same fluid network. This means that the operator may not need to ensure that unwanted or unneeded communication across different fluid networks is occurring.
Forming a wired network by connecting components of the fluid network by corresponding first and second lumens and first and second electrical connectors means that the network topology of the electrical network automatically matches that of the fluid network.
Pairing devices is easier.

Various aspects of the present disclosure will now be described with reference to the figures. For the purposes of illustration, components depicted in the figures are not necessarily drawn to scale. Instead, emphasis is placed on highlighting the various contributions of the components to the functionality of various aspects of the present disclosure. A number of possible alternative features are introduced during the course of this description. It is to be understood that, according to the knowledge and judgment of persons skilled in the art, such alternative features may be substituted in various combinations to arrive at different embodiments of the present disclosure.

### Fixed Skirt Embodiment

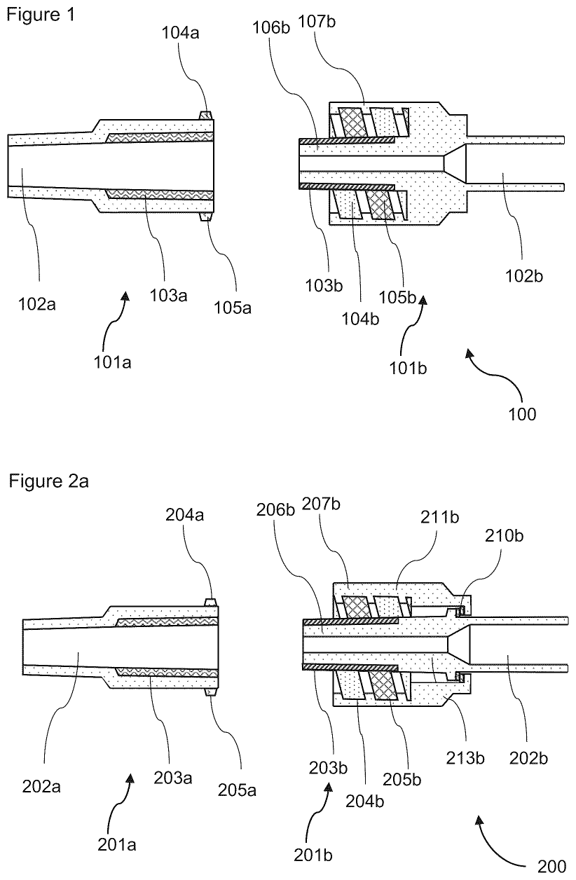

FIG. 1 shows an unconnected intravenous line connector assembly 100 comprising:
a first connector 101a comprising a first lumen 102a and one or more electrical first terminals 103a, 104a, 105a; and
a second connector 101b comprising a second lumen 102b and one or more electrical second terminals 103b, 104b, 105b,
wherein when the first connector 101a is connected to the second connector 101b, the first lumen 102a is connected to the second lumen 102b to allow fluid communication between the first and second connectors, and the first terminals 103a, 104a, 105a are connected with the second terminals 103b, 104b, 105b to allow electrical signals to pass between the first and second connectors.

In this case, each first terminal 103a, 104a, 105a is configured to connect with a corresponding second terminal 103b, 104b, 105b.

In this case, the first connector 101a is in the form of a tube which has a lumen 102a passing from one end of the connector to the other. The interior of the tube has a narrower end for attaching to an IV line, and a wider end for engaging with and attaching to the second connector. In this case, the narrowing of the tubes is consistent with Luer taper for forming a Luer lock.

The first connector in this case has three first terminals: a positive terminal 103a, a negative terminal 104a, and a data terminal 105a. The positive and negative first terminals 103a, 104a are configured to convey power. It will be appreciated that other terminals may be used including one or more of: a live terminal and a ground terminal.

Electrical cables (not shown) are used to connect to each of the first and second terminals. These electrical cables will allow the signals to be connected to electrical cables in the connected IV lines. The connector electrical cables may be housed within the body of the connectors to avoid shorting. The body of the connectors may be formed from electrically insulating materials. The electrical cables of the connectors, in this case, are connected to electrical cables housed within IV lines connected to the connectors. A first IV line is connected to the first connector, and a second IV line is connected to the second connector. This allows the cables of the first and second IV lines to be connected when the first and second connectors are connected. This allows power and data signals to be transmitted between the first and second IV lines across the intravenous line connector assembly 100.

In this case, the positive and negative first terminals 103a, 104a are formed as projections on the outside of the first connector. The data terminal 105a is formed around the inside of the tube around the lumen of the first connector. In other embodiments, other permutations of these terminals may be used (e.g., the negative terminal may be the terminal inside the tube).

The second connector 101b also comprises a tube forming a lumen 102b which extends from one end of the connector to the other.

In this case, the second connector 101b comprises two axial projections 106b, 107b. The lumen projection 106b closer to the lumen axis is configured to be inserted into the tube of the first connector 101a. On the outer surface of the lumen projection is a data second terminal 103b configured to connect to the data first terminal 103a of the first connector 101a.

In this embodiment, the first and second data terminals 103a,b are rotationally invariant about the lumen axis. This means that the first and second data terminals 103a,b can be rotated with respect to each other while maintaining connection.

A screw projection 107b on the second connector is positioned further away from the lumen axis and is configured to form a screw recess between the screw projection 107b and the lumen projection 106b for receiving the end of the first connector tube and the positive and second terminals 104a, 105a.

In this case, the inner surface of the screw projection 107b comprises screw threads with two starts (i.e., number of separate helical grooves in the female screw thread, or ridges in the male screw thread), each start being configured to receive one of the projecting terminals (the positive and negative terminals in this case). For clarity, the two starts are shaded differently, one with crosshatches and one with dots. It will be appreciated that the number of starts may correspond to the number of terminals to be received. It will be appreciated that the screw threads in this case are one example of complementary engagement surfaces used to physically engage and connect the first and second connectors securely together.

In some embodiments, the starts may have a different size or shape to restrict the engagement of the first and second screw terminals to one configuration. This may help ensure that a particular first terminal can only be connected to a corresponding second terminal. In some embodiments, the screw may be located on the outside of the lumen projection.

In this embodiment, neither of the first and second connectors have any moving parts. This means that they are robust and can be used to provide a secure wired electrical connection.

In this embodiment, the connector is configured to connect two IV lines to each other to form a fluid connection in which fluid can pass from a first IV line to a second IV line via the intravenous line connector assembly, and an electrical connection in which power and data signals can pass from the first IV line to the second IV line. This allows a wired electrical network to be established which corresponds to the fluid network. This reduces the number of lines required to do this as separate fluid and electrical lines are not required.

### Rotating Skirt Embodiment

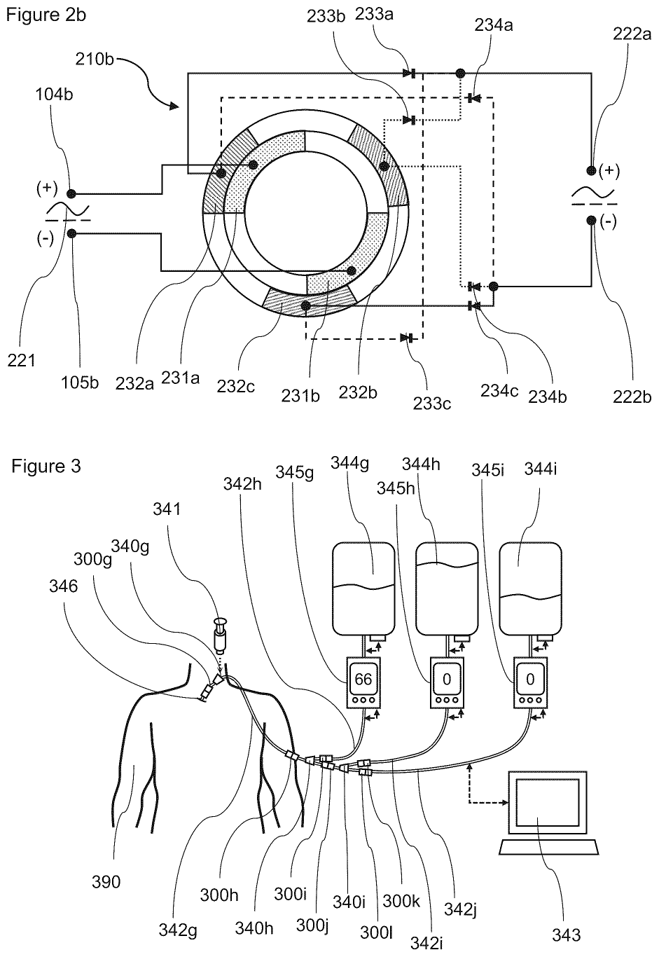

FIG. 2 shows a further embodiment of an unconnected intravenous line connector assembly 200 comprising:
a first connector 201a comprising a first lumen 202a and one or more electrical first terminals 203a, 204a, 205a; and
a second connector 201b comprising a second lumen 202b and one or more electrical second terminals 203b, 204b, 205b,
wherein when the first connector 201a is connected to the second connector 201b, the first lumen is connected to the second lumen to allow fluid communication between the first and second connectors, and the first terminals are connected with the second terminals to allow electrical signals to pass between the first and second connectors.

In this case, each first terminal 203a, 204a, 205a is configured to connect with a corresponding second terminal 203b, 204b, 205b.

In this case, the first connector 201b is the same as the first connector in the embodiment of FIG. 1.

Also like the embodiment of FIG. 1, the second connector 201b in this embodiment comprises a tube forming a lumen 202b which extends from one end of the connector to the other.

However, unlike the embodiment of FIG. 1, in this embodiment, the second connector comprises a rotatable sleeve 211b which is rotatable independently of the lumen 202b, the rotatable sleeve comprising engagement surfaces for physically connecting with the other connector.

In this case, the second connector comprises two projections. The lumen projection 206b closer to the lumen axis is configured to be inserted into the tube of the first connector. On the outer surface of the lumen projection is a second data terminal 203b configured to connect to the first data terminal 203a of the first connector.

In this embodiment, the first and second data terminals are rotationally invariant about the lumen axis. This means that the first and second data terminals can be rotated with respect to each other while maintaining connection.

A screw projection is formed by the rotatable sleeve 211b, which forms a screw projection 206b on the second connector 201b and is positioned further away from the lumen axis than the lumen projection. A screw recess is formed between the screw projection 206b formed by the rotatable sleeve 211b and the lumen projection 206b for receiving the end of the first connector tube and the positive and second terminals 204a, 205a.

In this case, the inner surface of the rotatable sleeve screw projection 207b comprises screw threads with two starts (i.e., number of separate helical grooves in the female screw thread, or ridges in the male screw thread), each start being configured to receive one of the projecting terminals (the positive and negative terminals in this case). It will be appreciated that the number of starts may correspond to the number of terminals to be received. It will be appreciated that the screw threads in this case are one example of complementary engagement surfaces used to physically engage and connect the first and second connectors securely together.

The use of the rotatable sleeve which can be rotated around the lumen means that the second connector can be connected to the first connector using the screw threads without rotating the tube or lumen of the first connector with respect to the tube or lumen of the second connector. This may help prevent kinking in the fluid line.

To facilitate connection between the channel body 213b and the rotatable sleeve 211b, there is a rotating connector assembly 210b. It will be appreciated that there are electrical cables (not shown) connecting the second terminals 204b, 205b on the rotating sleeve 211b to the rotating connector assembly 210b. Likewise, the in the channel body 213b, there is electrical cables (not shown) taking the electrical signals from the rotating connector assembly 210b.

As shown in FIG. 2b, the rotating connector assembly 210b, which allows an electrical signal to be connected regardless of the relative rotation between the channel body 213b and the rotatable sleeve 211b, comprises:
a first rotating connector comprising a plurality of discrete first terminals 231a-b spatially distributed around an axis of a circularly symmetric first surface, the discrete first terminals forming discrete first terminal pairs whereby a potential difference can be applied between each first terminal pair;
a second rotating connector having a plurality of discrete second terminals 232a-c spatially distributed around a circularly symmetric second surface, each of the discrete second terminals:
   being connected to a common outflow point 222a with an outflow diode 234a-c configured to allow electric current to flow from the discrete second terminal to the outflow point, and
   being connected to an inflow point 222b with an inflow diode 235a-c configured to allow electric current to flow from the inflow point to the discrete second terminal;
wherein the number of discrete first terminals does not equal the number of discrete second terminals such that, when the first and second surfaces are connected, regardless of the rotational orientation of the first surface with respect to the second surface:
   at least one pair of discrete first terminals are electrically connected to the inflow and outflow points; and
   no pair of discrete first terminals are electrically connected to the same discrete second terminal.

This allows a signal 221 to be transmitted from input terminals 104b, 105b to output terminals 222a-b regardless of the relative rotation of the sleeve with respect to the lumen.

For clarity, in FIG. 2b, the first and second rotating connectors are shown with one within the other. As shown in FIG. 2a, the rotating connectors are positioned on opposing surfaces which are transverse to the rotation axis. This means that when the engagement surfaces are tightened, the opposing surfaces are pushed together to improve the electrical connection through the rotating connector assembly.

As before, in other embodiments, the starts may have a different size or shape to restrict the engagement of the first and second screw terminals to one configuration. This may help ensure that a particular first terminal can only be connected to a corresponding second terminal.

In this embodiment, the first connector does not have any moving parts. The second connector is formed using two parts, the tube and the rotatable sleeve.

In this embodiment, the connector is configured to connect two IV lines to each other to form a fluid connection in which fluid can pass from a first IV line to a second IV line via the intravenous line connector assembly, and an electrical connection in which power and data signals can pass from the first IV line to the second IV line. This allows a wired electrical network to be established which corresponds to the fluid network. This reduces the number of lines required to do this as separate fluid and electrical lines are not required.

### IV Line Network

FIG. 3 is a front view of an embodiment of an intravenous line network comprising:
multiple intravenous lines 352g,h,l, each intravenous line comprising at least one electrical wire;
an intravenous line connector assembly 300g,h,l,j,k (e.g. embodiments as shown in FIGS. 1 and/or 2a) connecting the multiple intravenous lines; and
electrical equipment configured to interact with the fluids passing through the multiple intravenous lines.

In this case, the network comprises three bag IV lines 342h,l,j, each bag IV line being connected to a bag of fluid 342g,h,i. Each bag line feeds into a common cannula IV line 342g via two Y-branch connectors 340i,j. The cannula line comprises an injection port 340g for allowing the contents of a syringe 341 to be injected into the cannula 346 close to the cannula. Each of the IV lines are connected to the Y-branch connectors via two-line intravenous line connectors 300h,l,j,k as described above. Likewise, the cannula line is connected to the cannula via an intravenous line connector 300g as described above.

It will be appreciated that other embodiments of the intravenous line connector may have more than two connections. For example, the intravenous line connector may be configured to connect to three lines (e.g., a Y-branch connector) or more than three (e.g., a manifold connector).

In this embodiment, each of the IV lines are configured to carry electrical signals in addition to fluid. Electrical cable is configured to be attached to or embedded in the walls of the IV lines. It will be appreciated that each Y-branch connector may be configured to act as a splitter to allow electrical signals to connect to all of the various branches of the IV-line network.

In this embodiment, electrical equipment comprises:
a respective fluid data source associated with each fluid source (including syringe 341 and fluid bags 344g,h,i), the fluid data source being configured to transmit fluid identity data identifying the respective fluid; and
a controller 343 configured to receive and collate the fluid identity data transmitted from each data source via the electrical wires in the intravenous lines and the electrical connections provided by the intravenous line connector assemblies.

The controller in this case is a central computer attached to the electrical network formed by the intravenous line network. The controller may have information relating to the patient 390 (e.g., blood type, allergies, medical diagnoses etc.). In this case, the controller is connected to the electrical network using a plug and socket connection or a wireless connection.

In this case, the fluid data source may be simply a chip storing information identifying the contents of the fluid. For example, the fluid data source may contain information on the type of fluid (e.g., blood, blood type, saline solution, medication etc.), and the concentration of the fluid.

In some embodiments, each of the multiple intravenous lines and/or intravenous line connector assemblies may be configured to send identifying data so that multiple connected components of the fluid network may be identified. The topology of the various components may also be determined by receiving information on which components are connected via the electrical connections. For example, information on that lines 342i and 342j are both connected to a common line by Y-connectors may be used to ensure that the total fluid flow from these two lines does not exceed the capacity of the downstream components of the fluid network.

### Fluid Information

As the fluid data source is connected to the electrical network, the fluid data source may be configured to send a signal to the controller identifying itself. This may happen repeatedly until the controller responds with a confirmation of receipt. This allows the controller 343 to be aware of all the components of the network as they are connected.

The controller 343 may be configured to communicate with the electrical components of the network on a schedule set by the controller. This may help allow data to be transmitted along common lines sequentially. The controller may communicate with electrical components of the network using unique identifiers so that commands can be sent to all electrical components along the common network, but only be implemented by the correct electrical component.

In this embodiment, the controller 343 is configured to identify whether or not the fluids are compatible based on the information received from each of the fluid data sources.

In this case, the first bag and the second bag contain two different drugs, and the third bag contains a saline solution. In this case, the controller identifies that the two drugs in the first bag and the second bag are incompatible. That is, if mixed outside the body, components of at least one will form a precipitate. The controller is configured to identify the incompatibility based on a table of incompatibilities stored on memory within the controller.

In this embodiment the controller is configured, in response to identifying an incompatibility between two or more of the fluids, to create a flushing protocol to avoid the incompatible fluids coming in contact within the intravenous line network. In this embodiment, the flushing protocol is configured to ensure that after finishing a dose of either of the two drugs, a flush from the saline solution is injected through the network to clear the common cannula IV line before the other drug is connected.

To implement the protocol, in this embodiment, instructions are sequentially sent to control each of the three pumps 345g,h,l which control flow from each of the three bags.

In other embodiments, the controller may also be configured to identify incompatibilities with the patient (also known as contraindications) by identifying if any of the drugs would elicit a reaction from the patient based on the data from the patient stored on the controller.

### Network Monitoring

As noted above, the controller is configured to receive information from the fluid source after the fluid source is connected to the network. Likewise, other components of the network may also comprise a data source configured to identify itself to the controller when it is first connected to the network. For example, the cannula may comprise a cannula data source configured to send one or more signals to the controller.

In response to detecting a new component in the network, the controller may be configured to record a time at which one or more of the components of the intravenous line network is first connected. Based on the time elapsed between first connection and the current time, the controller may calculate the age of the components, the age being the time that the component has been used in the network. The controller may then be able to identify components of the network which are getting close to the end of life by, for example, comparing the determined age with a stored lifespan associated with that component. For example, the lifespan of a particular peripheral intravenous cannula may be 100 hours. The controller may be configured to provide a notification when this intravenous cannula is approaching its lifespan to allow medical professionals to change the cannula at the right time, and avoid complications due to exceeding the lifespan of the component.

As noted above, the electrical equipment in this case comprises multiple pumps delivering fluid from multiple fluid sources. The controller may be configured to switch between pumps and fluid sources based on when the fluid source is exhausted. For example, the patient may be receiving parenteral nutrition which requires multiple bags. The multiple bags may be set up and branched into a common line. The controller may be configured to control the pump to pump one bag into the patient first, and when that bag is finished, stop that pump and start the pump on the second bag to allow the nutrition bags to be injected sequentially. It will be appreciated that the flow may be controlled by controlling pumps and/or electronically controlled clamp (e.g., roller clamp). Clamps may be used to control gravity fed fluids.

### Alarms

Another major issue with IV systems is dealing with alarms. Alarms may be generated when a pump detects air in the line or unexpected blockages (e.g., kinks in the line). Due to the risks to the patient associated with these difficulties, the alarms are generally configured to be very sensitive and so many false alarms may be generated. This is problematic for medical personal, particularly if there is an onerous protocol for entering the patient's room (e.g., a masking and cleaning protocol for COVID-19) and the alarm can be determined to be false from the user's current location. The wired network allows the controller to be located outside the patient's room and at least some of the alarms to be dealt with outside the patient's room. In this case, the controller is configured to allow alarms to be acknowledged and cancelled remotely.

### Single Signal Embodiment

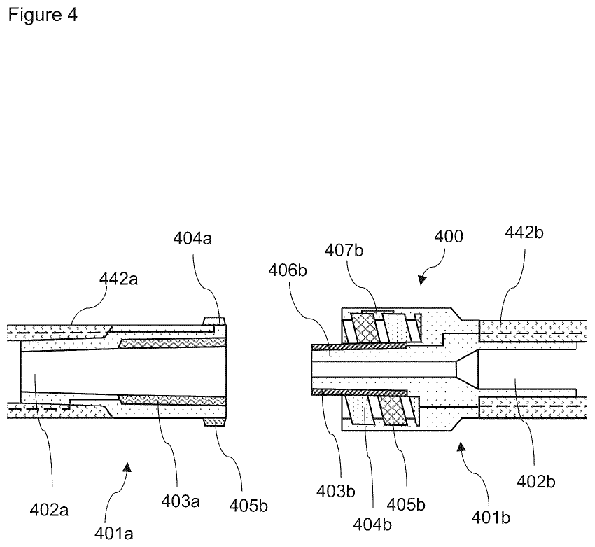

FIG. 4 shows a further unconnected intravenous line connector assembly 400 comprising:
a first connector 401a comprising a first lumen 402a and one or more electrical first terminals 403a, 404a, 405a; and
a second connector 401b comprising a second lumen 402b and one or more electrical second terminals 403b, 404b, 405b,
wherein when the first connector 401a is connected to the second connector 401b, the first lumen 402a is connected to the second lumen 402b to allow fluid communication between the first and second connectors, and the first terminals 403a, 404a, 405a are connected with the second terminals 403b, 404b, 405b to allow electrical signals to pass between the first and second connectors.

This embodiment is similar to the embodiment of FIG. 1, except that the two of the first terminals 404a, 405a are configured to carry the same electrical signal.

Like previous embodiments, each first terminal 403a, 404a, 405a is configured to connect with a corresponding second terminal 403b, 404b, 405b.

In this case, the first connector 401a is in the form of a tube which has a lumen 402a passing from one end of the connector to the other. The interior of the tube has a narrower end for attaching to an IV line 442a, and a wider end for engaging with and attaching to the second connector. In this case, the narrowing of the tubes is consistent with Luer taper for forming a Luer lock (or a luer slip).

In this case, there are three first terminals 403a, 404a, 405a and three second terminals 403b, 404b, 405b. In this case, one first terminal 403a is positioned on a surface facing the inside of the tube towards the lumen and two first terminals 404a, 405a is positioned on the outside of the tube away from the lumen. However, unlike the embodiment of FIG. 1, the two outer first terminals are electrically connected to each other. The connection may be by a cable connecting the two first terminals; extending the exposed surfaces of the terminals to that they come into contact (e.g., effectively forming a single larger terminal), or by delivering the same signal to both terminals independently using separate cables.

It will be appreciated that, in other embodiments, one of these duplicated terminals may be omitted or replaced by a non-connected component of the same shape (i.e., to facilitate physical connection in the same way, but not electrical connection). However, using two terminals instead of one may improve the electrical connection.

A single signal connector may be used to transfer a power signal or a data signal.

Electrical cables are used to connect to each of the first and second terminals to convey the signal to, and along, the attached IV line. The electrical cables may be housed within the body of the connectors to avoid shorting. The body of the connectors may be formed from electrically insulating materials. The electrical cables of the connectors, in this case, are connected to electrical cables housed within IV lines connected to the connectors. A first IV line 442a is connected to the first connector, and a second IV line 442b is connected to the second connector. This allows the cables of the first and second IV lines to be connected when the first and second connectors are connected. This allows power and/or data signals to be transmitted between the first and second IV lines across the intravenous line connector assembly 400.

In this case, first terminal 404a is formed as a threaded projection on the outside of the first connector.

The second connector 401b also comprises a tube forming a lumen 402b which extends from one end of the connector to the other.

In this case, the second connector 401b comprises two axial projections 406b, 407b. The lumen projection 406b closer to the lumen axis is configured to be inserted into the tube of the first connector 401a. On the outer surface of the lumen projection is a data second terminal 403b configured to connect to the data first terminal 403a of the first connector 401a.

In this embodiment, the one set of connectable terminals 403a,b are rotationally invariant about the lumen axis. This means that the first and second data terminals 403a,b can be rotated with respect to each other while maintaining connection.

A screw projection 407b on the second connector is positioned further away from the lumen axis and is configured to form a screw recess between the screw projection 407b and the lumen projection 406b for receiving the end of the first connector tube and the positive and second terminals 403a, 404a.

In this case, the inner surface of the screw projection 407b comprises screw threads with two starts (i.e., number of separate helical grooves in the female screw thread, or ridges in the male screw thread). It will be appreciated that the screw threads in this case are one example of complementary engagement surfaces used to physically engage and connect the first and second connectors securely together.

In this embodiment, the two starts of the female threads form two separate terminals. In this embodiment, the two terminals are connected to each other via a cable within the body of the connector (shown as a solid line). It will be appreciated that in other embodiment where two first terminals carry the same signal, the corresponding second terminals may be combined to form an extended second terminal.

In this embodiment, neither of the first and second connectors have any moving parts. This means that they are robust and can be used to provide a secure wired electrical connection.

In this embodiment, the connector is configured to connect two IV lines to each other to form a fluid connection in which fluid can pass from a first IV line to a second IV line via the intravenous line connector assembly, and an electrical connection in which power and data signals can pass from the first IV line to the second IV line. This allows a wired electrical network to be established which corresponds to the fluid network. This reduces the number of lines required to do this as separate fluid and electrical lines are not required.

### Network Configuration Determination

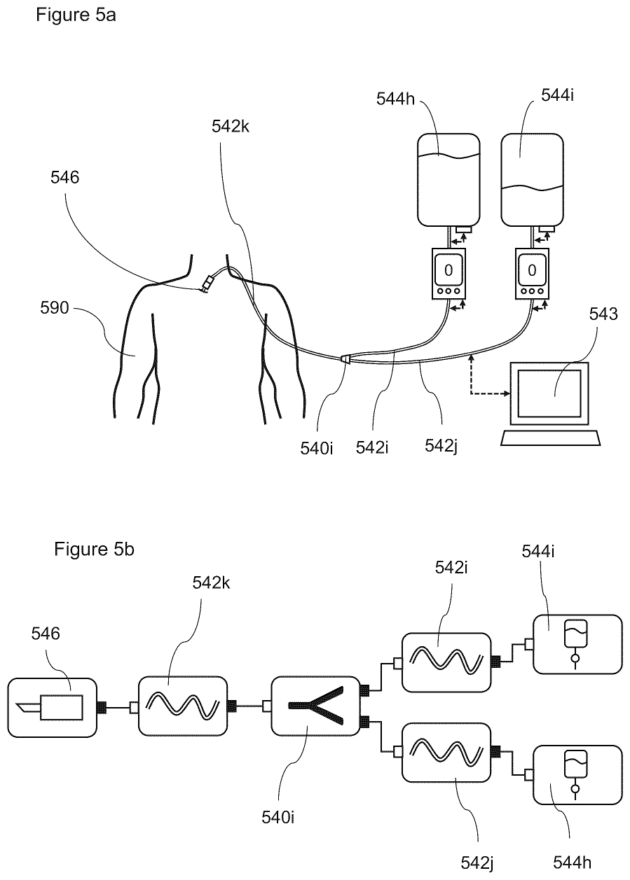

FIG. 5a shows an embodiment of an intravenous line network for providing fluids to a patient 590 including two fluid sources 544i,h connected to a cannula 546 via three fluid lines 542i,j,k and a Y-connector 540i. In this embodiment, the two pumps are considered as integral parts of the fluid sources. In other embodiments, the pumps may be separate intravenous components.

The system also comprises a controller 543 which receives and processes information from each of the electrically networked intravenous components. In this case, the controller is a separate computer. In other embodiments, the controller may form part of one or more of the components making up the fluid network.

In this embodiment, each of the components are connected to each other using connector assemblies such as those described in the present disclosure.

FIG. 5b is a schematic diagram of the network topology of the intravenous line network. In the network topology, each of the components are represented by a block with an image representing the type of component. Each block has one or more connectors corresponding to the number of connector assemblies that they can form with other components:
The cannula 546 and the two fluid sources 544i,h each have a single connector;
Each line 542i,j,k has two connectors; and.
The Y-connector has three connectors.

In this embodiment, a connector assembly is formed by connecting complementary connections together (e.g., male and female). In this case, the male connectors are indicated by white boxes and the female connectors by black boxes. The lines connecting a white and black box indicates that the connectors have formed a connector assembly to connect the two components. Each connected connector assembly allows for electronic connection via connected terminals and fluid transmission via connected lumens.

In this embodiment, each of the components has a data source which is configured to transmit identity data to the controller 543 using the wired networked formed by the components being connected together by the connector assemblies. In this case, the controller receives information indicating that there are three lines, two fluid sources, a Y-connector and a cannula within the electrical network.

From this information, the controller is able to determine the network topology of the network. It will be appreciated that there are a number of constraints which the controller may use in determining the network topology. For example, the controller may limit possible solutions using one or more of the following:
The determined network must be a single interconnected network comprising all the received components. E.g., connecting a fluid source directly to the cannula is physically possible, but this can be ruled out because it would mean that none of the other components could be connected to this network because there would be no other free connectors.
The determined network must permit flow from each of the fluid sources to the fluid exit (in this case, the cannula).
Every connection between two components must comprise two complementary connectors.
Constraints on the number of connectors for each component which must be attached. For example, a line may require that both connectors are connected to another components, whereas a Y connector may require an outlet and at least one inlet connector to be connected to another component.

The identity of the components may also introduce other constraints. For example, a particular type of cannula may require that it is connected to a line, and not directly to a Y-connector, or a drip connector may require that it is connected directly to a fluid source. It will be appreciated that the components may be configured to send such information to the controller over the wired network, or that the controller may be configured to determine this information based on the identified type of component (e.g., using information that is pre-stored on the controller).

Based on the constraints of the network, the controller determines the network topology shown in FIG. 5b. The two fluid sources are each connected to a respective line which are, in turn, connected to a Y-connector. The Y-connector feeds into the third line and on into the cannula which is inserted into the patient. In other embodiments, the components may be configured to communicate and determine the components with which they form a direct connection.

In this way, the controller can self-monitor the configuration of the fluid network.

In this embodiment, all the three lines are considered to be identical by the controller. It will be appreciated that, in other embodiments, the three lines may be different (e.g., a different bore), or be distinguishable by having different unique identifiers (e.g., which could be used to determine age).

The network may be configured to be monitored for changes to the network topology. For example, the controller may be configured to periodically poll the components to ensure that the identity of the network components is the same as that of the determined network topology. For example, if a fluid source is replaced, removed or added, it may be important for the system to recognise this change. Other embodiments may be configured to identify changes by one or more of: periodically transmitting identify information to the wired network and/or identity data being transmitted in response to two connectors being connected or separated.

In other embodiments, the controller may be configured to determine how fluids would flow through the network using the type of components and the determined network topology. For example, the controller may be configured to determine the volume of flush required to flush the common line so that incompatible fluids are not mixed within the common line. The controller may be configured to determine a measure of mixing where multiple fluids are injected into a common line simultaneously. This determination may take into account the length and/or bore of the common line, the rate of injection of each of the multiple fluids, the volume of each of the multiple fluids injected, and the physical characteristics of the multiple fluids (e.g., viscosity, miscibility, density etc.).

It will be appreciated that using a wired network to determine the network topology of the electrical and fluid network formed by the intravenous components may have advantages over determining wirelessly which components are in the vicinity, because wireless identification may detect other intravenous components which are not connected to the network (e.g., forming a separate independent network).

It will be appreciated that the system may allow the network to be controlled based on the determined topology.

For example, the controller may control the two pumps forming part of the two fluid sources 544i,j. Control of electrical equipment interacting with the fluid network and/or patient may be performed by sending signals to the electrical components via the wired network formed through the connected connector assemblies or by sending control signals via an independent second network (e.g., a wireless network).

The control of the pumps may be adjusted, based on the determined topology to achieve particular aims including, for example, ensuring that the total flow from the two fluid sources does not exceed the flow capacity of the common fluid line 542k (or other downstream network components); if the fluids are incompatible, ensuring that the common line 542k is adequately flushed when swapping between the two fluid sources; and, if the fluids are incompatible, adjusting the pumping rates to ensure that the fluids are adequately mixed in the common line before entering the patient.

### Other Options

The wired electrical network may be configured to communicate with one or more other networks. Multiple wired networks according to the present technology may communicate when connected to the same patient. This communication may be used to ensure that multiple drugs delivered to the same patient via separate cannulas can be coordinated. E.g., if a patient is connected to one cannula delivering epinephrine and a separate cannula delivering vasopressin (both of which affect blood pressure), the flow of these drugs should not be simultaneously adjusted to achieve a target blood pressure. Communication between the two wired electrical network may allow the vasopressin flow to be fixed while the epinephrine is adjusted.

In other embodiments, the intravenous line connector assembly may have only two electrical first terminals and two electrical second terminals. For example, the first connector may comprise one first terminal on the inside of the tube, and another first terminal on the outside of the tube. Such an arrangement would allow either power or data to be transmitted at a particular time.

In other embodiments, the complementary engagement surfaces may be separate from the terminals. That is, the complementary engagement surfaces may be configured just to ensure a physical connection between the two terminals, while the electrical connection is provided by other components of the connectors.

In other embodiments, the controller may be configured to detect when a manually activated fluid source, such as a syringe, is connected to the intravenous line network. The controller may be configured to instruct the pumps connected to the same IV network to stop in response to detecting that a manually activated fluid source. This ensures that cannula is not overwhelmed with a flow rate that is two high.

A syringe may be configured to generate a signal to be communicated to the controller.

In other embodiment, the electrical equipment comprises a cannula data source configured to communicate the size of a lumen to the controller. Based on this the controller can adjust the flow rates being delivered by multiple fluid sources such that the total flow rate delivered to the lumen is below a predetermined threshold. For example, if the bags of FIG. 3 instead held fluids which were to be injected simultaneously (i.e., with at least two of the pumps operating at the same time), an excess of pressure may be built up within the cannula line if the total pumping rate exceeded the capacity of the cannula.

Although the present disclosure has been described and illustrated with respect to preferred embodiments and preferred uses thereof, it is not to be so limited since modifications and changes can be made therein which are within the full, intended scope of the present disclosure as understood by those skilled in the art.

What is claimed is:

1-20. (canceled)

21. An intravenous line connector assembly comprising:
a first connector comprising a first lumen and one or more electrical first terminals; and
a second connector comprising a second lumen and one or more electrical second terminals,
wherein when the first connector is connected to the second connector, the first lumen is connected to the second lumen to allow fluid communication between the first and second connectors, and the first terminals are connected with the second terminals to allow electrical signals to pass between the first and second connectors.

22. The intravenous line connector assembly according to claim 21, wherein the first and second connectors comprise respective complementary engagement surfaces configured to physically maintain connection between the first and second connectors.

23. The intravenous line connector assembly according to claim 22, wherein the complementary engagement surfaces comprise screw threads.

24. The intravenous line connector assembly according to claim 23, wherein the screw threads comprise multiple thread starts, each start providing a separate said first terminal.

25. The intravenous line connector assembly according to claim 21, wherein one of the first and second connectors comprise a rotatable sleeve which is rotatable independently of the lumen, the rotatable sleeve comprising engagement surfaces for physically connecting with the other connector.

26. The intravenous line connector assembly according to claim 21, wherein at least one of the first terminals is formed on a surface which is circularly symmetric about a lumen axis.

27. The intravenous line connector assembly according to claim 21, wherein the intravenous line connector assembly comprises two electrical first terminals and two electrical second terminals.

28. The intravenous line connector assembly according to claim 21, wherein the intravenous line connector assembly comprises three electrical first terminals and three electrical second terminals.

29. The intravenous line connector assembly according to claim 21, wherein at least one of the first and second connectors has no moving parts.

30. The intravenous line connector assembly according to claim 21, wherein the electrical signals comprise power signals.

31. The intravenous line connector assembly according to claim 21, wherein the electrical signals comprise data signals.

32. An intravenous line network comprising:
multiple intravenous components including at least one intravenous line, each intravenous component comprising at least one electrical wire;
the intravenous line connector assembly according to claim 21 connecting the multiple intravenous components; and
electrical equipment electrically connected to at least one of the electrical wires.

33. The intravenous line network according to claim 32, wherein the multiple intravenous components comprise multiple fluid sources connected to a common intravenous line, and wherein the electrical equipment comprises:
a respective data source associated with each fluid source, the data source being configured to transmit fluid identity data identifying the respective fluid; and
a controller configured to receive the fluid identity data transmitted from each data source via the electrical wires in the intravenous lines and the electrical connections provided by the intravenous line connector assemblies.

34. The intravenous line network according to claim 33, wherein the controller is configured to identify whether the fluids are compatible or incompatible.

35. The intravenous line network according to claim 34, wherein the controller is configured, in response to identifying an incompatibility between two or more of the fluids, to create a flushing protocol to avoid the incompatible fluids coming in contact within the intravenous line network.

36. The intravenous line network according to claim 32, wherein the electrical equipment comprises a controller, the controller being configured to:
record a time at which one or more of the multiple intravenous components of the intravenous line network is first connected, and
calculate an age of the components based on the time elapsed between first connection and a current time.

37. The intravenous line network according to claim 32, wherein the electrical equipment comprises a controller, and multiple pumps delivering fluid from multiple fluid sources, the controller being configured to switch between pumps and fluid sources based on when the fluid source is exhausted.

38. The intravenous line network according to claim 32, wherein the electrical equipment comprises a controller and a data source configured to communicate a size of a lumen to the controller, the lumen receiving fluids from multiple fluid sources, the controller configured to adjust flow rates being delivered by multiple fluid sources such that a total flow rate delivered to the lumen is below a predetermined threshold.

39. The intravenous line network according to claim 32, wherein the electrical equipment comprises a controller, the controller being configured to: receive data via the electrical wires relating to a type of the multiple intravenous components; and, based on the received intravenous component types, determine a network topology for a fluid network.

40. The intravenous line network according to claim 32, wherein the electrical equipment comprises multiple pumps, and wherein at least one of the pumps is a controller pump having a controller configured to control at least one of the other said pumps.
